(19) United States
(12) Patent Application Publication
(10) Pub. No.: US 2023/0309880 A1
(43) Pub. Date: Oct. 5, 2023

(54) DEVICE FOR CONNECTING A CANNULA TO A CONTAINER

(71) Applicants: MEDIZINISCHE UNIVERSITÄT WIEN, Vienna (AT); TECHNISCHE UNIVERSITÄT WIEN, Vienna (AT)

(72) Inventors: David BARON, Vienna (AT); Christoph EISENMENGER-SITTNER, Gumpoldskirchen (AT); Martin MÜLLER, Vienna (AT); Lukas THAJER, Eichgraben (AT); Stefan PRIBITZER, Altlichtenwarth (AT)

(73) Assignees: MEDIZINISCHE UNIVERSITÄT WIEN, Vienna (AT); TECHNISCHE UNIVERSITÄT WIEN, Vienna (AT)

(21) Appl. No.: 17/923,253

(22) PCT Filed: May 3, 2021
(86) PCT No.: PCT/IB2021/053669

§ 371 Date: Nov. 4, 2022

(30) Foreign Application Priority Data
May 5, 2020 (AT) A 108/2020

(51) Int. Cl. A61B 5/154; A61B 5/15

(52) U.S. Cl. CPC A61B 5/154; A61B 5/15003; A61B 5/150221

(57) ABSTRACT

The invention relates to a device for connecting a cannula to a container under reduced pressure, in particular a blood culture flask, including an inflow opening for connecting to the cannula or to a connection tube connected to the cannula, an outflow opening for connecting to a hollow pin for piercing a closure of the container, and a tight fluidic connection of the inflow opening to the outflow opening. According to the invention, a reservoir which is separated from the fluidic connection and contains a specified gas quantity is connected to the fluidic connection with the interposition of a valve which is preferably open towards the fluidic connection.

### CROSS-REFERENCE TO RELATED APPLICATIONS

The present application is a national phase application of PCT Application No. PCT/IB2021/053669, filed May 3, 2021, entitled “DEVICE FOR CONNECTING A CANNULA TO A CONTAINER”, which claims the benefit of European Patent Application No. A 108/2020, filed May 5, 2020, each of which is incorporated by reference in its entirety.

### BACKGROUND OF THE INVENTION

### 1. Field of the Invention

The invention relates to a device for connecting a cannula to a container being under reduced pressure, in particular to a blood culture bottle, comprising an inflow opening for connection to the cannula or to a connecting tube leading to the cannula, an outflow opening for connection with a hollow mandrel for piercing a closure of the container, and a tight fluidic connection of the inflow opening with the outflow opening, as well as to a method of connecting a cannula to a container being under reduced pressure, in particular to a blood culture bottle.

### 2. Description of the Related Art

In the context of the present invention, a reduced pressure is understood to be a pressure that is below atmospheric pressure.

Devices of the type mentioned above are known and widely used in clinical practice for taking blood samples for diagnostic purposes. They are also known as adapters or luer adapters and are used to routinely draw blood from patients in order to subject the blood to regular checks or analyses. The blood is usually taken from a patient's blood vessel via the cannula. The cannula, which is inserted into the blood vessel, is usually connected to a blood culture bottle sealed by a membrane via a connecting tube, such as a so-called Heidelberg extension, and a special hollow mandrel made of plastic. The hollow mandrel can also be designed as a cannula, so that another cannula different from the aforementioned patient-side cannula can be part of the device according to the invention.

A blood culture bottle usually has a volume of about 80 ml, part of which is usually filled with nutrient solution. It is usually made of a plastic or other, usually transparent, dimensionally stable material and is evacuated up to a gas pressure of approx. 0.3 bar to 0.4 bar. The reduced pressure compared to atmospheric pressure is used to aspirate the patient's blood through the cannula after piercing the membrane with the mandrel in order to fill the blood culture bottle without further intervention. Blood is drawn from the blood vessel through the cannula and, if necessary, the connecting tube as well as through the hollow mandrel until pressure equalization has largely occurred, at which point the blood culture bottle contains approximately 20 ml to 30 ml of blood.

In everyday clinical practice, it is necessary, especially in multimorbid patients or patients with complex diseases or unclear symptoms, to perform certain blood tests repeatedly, whereby several different test series may have to be scheduled, which, in addition, often has to be performed several times a day. It is therefore not uncommon for some patients to have several 100 ml of blood drawn per day in this way for routine analyses, which by its very nature is a considerable burden that is poorly tolerated by patients with an already weakened constitution. In addition, a surgical procedure is often scheduled at the end of a longer examination and monitoring period, which in turn is usually associated with a not insignificant loss of blood. For this reason, patients are often supplied with blood units in parallel, which in turn is associated with certain risks (infection, influence on the immune system, overloading of the cardiovascular system, etc.) and leads to a shortage of blood units elsewhere.

This is all the more alarming because much smaller amounts of blood than the 20 ml to 30 ml mentioned would be sufficient for most of the blood tests for which blood is drawn from patients using the blood culture bottles mentioned. In particular, even from the point of view of increasingly modern laboratory medicine, it seems perfectly adequate to draw, for example, only 5 ml to 10 ml of blood per blood culture bottle. However, this often does not happen in everyday clinical practice, as the blood culture bottles are filled very quickly until the pressure is equalized, and special care would therefore be required on the part of the personnel, who are usually hardly aware of the problem described above, in order to actively limit the quantities taken by prematurely stopping the filling process.

### SUMMARY OF THE INVENTION

The present invention is therefore based on the task of further designing a device of the type mentioned above in such a way that, when the device according to the invention is used together with conventional blood culture bottles, only a reduced amount of blood is drawn compared to the use of a Luer adapter, in order to avoid or not further exacerbate anemic conditions in patients.

To solve this problem, a device of the type mentioned at the beginning is characterized according to the invention in that a reservoir that contains a predetermined quantity of gas and is separate from the fluidic connection is connected to the fluidic connection with the interposition of a valve which preferably opens towards the fluidic connection. By connecting a reservoir separate from the fluidic connection and containing a predetermined amount of gas, when the closure of the container, which is generally formed by a metal or plastic membrane, is pierced, the relatively strong negative pressure in the container or blood culture bottle, with the valve opening in the direction of the tight fluidic connection, draws a certain amount of gas from the reservoir into the connection, thereby reducing the negative pressure prevailing in the blood culture bottle. The valve then closes, removing the reservoir from the system, in order to allow blood sampling. As a result, only a smaller pressure difference is available for drawing blood through the cannula and, if necessary, the connecting tube, so that a smaller volume of blood is drawn into the blood culture bottle overall without the need for personnel to intentionally stop the flow of blood through the device. In this way, the risk of anemia can be reduced in patients who must undergo frequent blood draws, without requiring increased work or care on the part of medical personnel.

According to a preferred embodiment of the present invention, the outflow opening is integrally connected to the hollow mandrel. Thus, the present invention can be used on the outflow opening side, on the one hand, with a hollow mandrel or cannula separately fitted on or into the outflow opening for piercing the closure of the blood culture bottle or, on the other hand, in accordance with the preferred embodiment just described, with the hollow mandrel already formed on the outflow opening by the manufacturer. In this preferred way, a ready-to-use product, preferably sterile packaged, can be offered, which enables fast and particularly patient-friendly blood sampling.

It is obvious that the basic idea of the present invention of reducing the negative pressure in the blood collection system can be realized with a wide variety of reservoirs. For example, it would be conceivable to use a reservoir with a very small volume and a gas volume under high pressure. However, this is potentially problematic with regard to patient safety and therefore problematic in any case with regard to a clinical approval of the device according to the invention. For these reasons, with respect to the common sizes of the blood culture bottles in question and the common pressure levels in the blood culture bottles already mentioned at the beginning, it is preferred in the context of the present invention that the amount of gas in the reservoir is at atmospheric pressure and occupies a volume of 40 cm³ to 100 cm³, preferably 50 cm³ to 90 cm³, further preferably 60 cm³ to 80 cm³ and particularly preferably 70 cm³. The fact that the amount of gas in the reservoir is at atmospheric pressure eliminates the possibility that any circumstances could cause the gas to leak from the reservoir into a patient's bloodstream. At atmospheric pressure in the reservoir, the above volumes have been determined in experiments to be suitable for achieving a reduction in the negative pressure in a blood culture bottle to the degree that approximately 5 ml to 10 ml of blood is drawn into the blood culture bottle.

With respect to the control of the valve, according to a preferred embodiment of the present invention, it may be provided that the valve is switchable by the differential pressure between the reservoir and the fluidic connection. In this way, valve actuation is automatic and low-cost embodiments of the present invention can be realized.

Here, it is preferably provided that the valve can be opened by the negative pressure prevailing in the fluidic connection. This means that the valve, and in particular the valve closure member, is designed to be opened by the negative pressure and thus remain open until either complete pressure equalization has occurred or the valve closure member closes again due to a reset tendency before complete pressure equalization has occurred between the fluidic connection and the reservoir.

Alternatively or additionally, the invention may be further embodied in that the valve is closable by the positive pressure prevailing in the fluidic connection, in accordance with a preferred embodiment of the present invention. This means that the valve, and in particular the valve closure member, is designed in such a way that excess pressure in the fluidic connection, such as occurs when blood at ambient pressure flows into the fluidic connection, causes the valve closure member to close.

It is obvious that, from a constructive point of view, there are various ways to implement the inventive idea of the present invention. According to a preferred embodiment of the present invention, however, it is provided that a chamber is arranged on the side of the valve in communication with the fluidic connection, so that a positive pressure prevailing in the chamber acts on a valve closure member of the valve in the closing direction. In this case, the chamber provides the necessary space for the supply of gas from the reservoir as a result of the negative pressure prevailing in the container.

In a particularly preferred manner, the present invention is here further embodied in that the reservoir comprises a, preferably circular, perforated plate having at least one hole as a reservoir wall and the at least one hole is covered by the valve closure member of the valve in the closed position of the valve on the side of the chamber. This preferred configuration represents a particularly compact design in which a reservoir wall or wall of the reservoir simultaneously forms a valve element, the reservoir being connected simply via at least one hole in the perforated plate. Preferably, the perforated plate has a plurality of holes. The at least one hole is covered by the valve closure member in the closed position of the valve, synonymous with the closed position of the valve closure member, and is not released until a negative pressure is applied to the outflow opening to allow passage of a quantity of gas from the reservoir. This is due to the pressure difference that occurs between the tight fluidic connection and the reservoir when the closure of the container is pierced. The negative pressure in the blood culture bottle transfers to the tight fluidic connection or to the chamber via the outflow opening, so that there is positive pressure in the reservoir compared to the tight fluidic connection. This actuates the valve closure member, in the present preferred case the valve closure member is lifted from the at least one hole in the perforated plate and a certain amount of gas can pass from the reservoir into the tight fluidic connection or into the chamber and flow into the blood culture bottle via the outflow opening, so that a reduction in the negative pressure occurs here. After pressure equalization has taken place, whereby any restoring force of the valve closure member must also be taken into account, the valve closure member closes again, so that when the blood is drawn in via the inflow opening and the cannula into the blood culture bottle, no blood can enter the reservoir and only the relatively small volume of the chamber must be additionally filled with blood, which ultimately represents a dead volume in the blood drawing system according to the invention.

Further integration of the components and a user-friendly design that facilitates handling of the blood drawing device according to the invention is achieved in that the reservoir is formed as an annular chamber surrounding the fluidic connection, preferably with the perforated plate supporting the fluidic connection. The reservoir surrounds an inflow line leading from the inflow opening to the outflow opening and is preferably designed as a hollow cylinder, through whose reservoir wall opposite the perforated plate the inflow line is led to the connection for the cannula or a connecting tube. The inflow line is preferably arranged along an axis of rotation of the reservoir, thus it passes through the center of the reservoir. This allows the reservoir to be grasped and the hollow mandrel intuitively placed against the closure of the container to pierce it. In this way, the handling of the device according to the invention does not differ significantly from the use of a conventional Luer adapter, which is of great importance for the acceptance of the device according to the invention.

According to a preferred embodiment of the present invention, the valve closure member is formed as a circular, deformable disc with a valve hole, preferably formed in the center. The valve closure member is therefore largely similar to a conventional sealing ring, which covers the at least one hole in the perforated plate and is lifted off the at least one hole by the aforementioned pressure difference between the chamber and the reservoir, whereupon a quantity of gas escapes from the reservoir and into the chamber through the valve hole in the valve closure member.

With respect to the shape of the chamber and its interaction with the valve closure member, the present invention is preferably further configured in that the perforated plate and a wall of the chamber opposite the perforated plate bear against the valve closuring closure member at the edge thereof, preferably in a clamping manner, and in the region of the valve hole the wall of the chamber opposite the perforated plate is spaced from the valve closure member. Due to the preferred clamping contact of the perforated plate and the wall of the chamber opposite the perforated plate, the valve closure member is suitably supported in the chamber without the need for any other installations in the chamber and without the need for any fasteners. The fact that in the area of the valve hole the wall of the chamber opposite the perforated plate is spaced from the valve closure member creates space for the action of the valve closure member to open the valve during pressure equalization. The wall of the chamber opposite the perforated plate is preferably spaced in such a way that just enough space is created for the action or the release movement of the valve closure member, so that the chamber is designed to be as small as possible in terms of its volume. This is useful because during the blood collection following pressure equalization, blood flows through the chamber at the transition from the inflow opening or inflow line to the outflow opening or outflow line, and at the end of the blood collection, the volume of the chamber does not empty into the container or blood culture bottle, so that the volume of the chamber is to be regarded as dead volume. This dead volume should of course be kept as small as possible with regard to the task of the present invention.

Since in the present invention the valve is to be particularly inexpensive and therefore simple and composed of few parts, the opening pressure of the valve is preferably to be determined solely by the inherent resistance of the valve closure member or by its strength or deformability. To this end, the valve closure member according to a preferred embodiment of the present invention comprises a material selected from the group consisting of rubber, silicone and polyolefins. These materials can be processed cheaply and in mass production, or can be selected from commercially available sealing rings to provide a valve closure member for use with a device according to the present invention. The above materials offer excellent sealing properties for the present application and can be selected or manufactured in suitable thickness for the suitable strength mentioned.

Preferably, the present invention is further configured in that the valve is configured to open at a negative pressure of the fluidic connection relative to the reservoir of 0.3 bar to 0.8 bar, preferably 0.4 bar to 0.7 bar, more preferably 0.5 bar to 0.6 bar. It is further preferred that the valve is configured to close at an overpressure of the fluidic connection relative to the reservoir of 0.1 bar to 0.4 bar, preferably 0.2 bar to 0.3 bar, more preferably 0.25 bar. These preferred ranges of the pressure difference between the inflow opening and the outflow opening ensure, depending on the sizes of the target containers or blood culture bottles customary in clinical operation and the cannulas customarily used, that only the volumes of blood mentioned at the beginning are actually drawn into the blood culture bottle.

The method according to the invention for connecting a cannula to a container being under reduced pressure, in particular to a blood culture bottle, comprises the steps:
providing a connecting device for connecting the cannula to the container being under reduced pressure, in particular a connecting device as described above,
connecting the connecting device on the one hand to the cannula and on the other hand to the container that is under reduced pressure, thereby establishing a fluidic connection from the cannula to the container, drawing a quantity of gas from a reservoir of the connecting device, which is separate from the fluidic connection, into the container, said drawing being effected by means of a negative pressure prevailing in the container relative to the reservoir.

By drawing a quantity of gas into the container from a reservoir of the connecting device that is separate from the fluidic connection, the negative pressure prevailing in the blood culture bottle is reduced. As a result, only a smaller pressure difference is available for drawing blood through the cannula and, if necessary, the connecting tube, so that a smaller volume of blood is drawn into the blood culture bottle overall without the need for personnel to intentionally stop the flow of blood through the device. In this way, the risk of anemia can be reduced in patients who must undergo frequent blood draws, without requiring increased work or care on the part of medical personnel.

Preferably, the method according to the invention is further embodied in that the drawing of the gas volume from the reservoir into the container is performed until a complete or partial pressure equalization between the reservoir and the container.

Preferably, the drawing of the gas volume from the reservoir into the container is performed via a valve arranged between the reservoir and the fluidic connection. The valve closes the sealing connection against the reservoir after the gas is drawn from the reservoir, so that no blood can enter the reservoir during subsequent blood collection.

Preferably, the method according to the invention is further embodied in that the reservoir is filled with a predetermined quantity of gas, which is dimensioned such that the pressure prevailing in the container is increased by 30%400%, preferably 40%-90%, more preferably 50%-80%, more preferably 60%-70%, as a result of the pressure equalization. In this way, the negative pressure in the blood culture bottles is reduced to such an extent that the target quantities of blood mentioned at the beginning can be drawn.

This is particularly successful if a predetermined amount of gas is provided in the reservoir at atmospheric pressure and with a volume of 40 cm³ to 100 cm³, preferably 50 cm³ to 90 cm³, further preferably 60 cm³ to 80 cm³ and particularly preferably 70 cm³.

The following describes how to determine the size of the volume of the reservoir by which the gas pressure in the blood culture bottles can be increased so that a smaller volume of blood is drawn into the blood culture bottle overall. It should be noted that these theoretical considerations may not be reproduced unchanged in practice due to a restoring force of the valve closure member, but these calculations provide the computational underpinning for the dimensioning of the reservoir or for the amount of gas in the reservoir.

The initial gas pressure in a blood culture bottle is denoted by p₁ in the following. A volume of liquid V_{b} is drawn into the blood culture bottle as soon as the hollow mandrel or a cannula is pierced through the closure of the blood culture bottle, which is connected in an airtight manner, for example, to a tube whose other end is inserted, for example, into a blood vessel. It is essential that the pressure p₂ acting outside the blood culture bottle is greater than the gas pressure inside the blood culture bottle. In its intended use for blood sampling, it can be assumed that p₂, when blood is drawn from a blood vessel, is approximately equal to the air pressure p₁. Even if the liquid is taken from an open glass, as shown in the figures discussed below, p₂ is equal to the air pressure. Based on the gas equation for ideal gases

P*V=n*R*T,

where R is the general gas constant, p is the gas pressure, V is the volume of the gas, and n is the amount of substance in the gas, the internal pressure in a blood culture bottle can be calculated as follows. In the original state, there is a certain amount of liquid in the blood culture bottle. The volume of the gas in the blood culture bottle is denoted by V₁. The following therefore applies before filling the bottle:

p₁*V₁=n₁*R*T

Then the blood culture bottle, due to the negative pressure prevailing inside, is filled with a liquid, for example blood from a blood vessel, with the liquid volume V_{b}, so that subsequently the internal pressure in the blood culture bottle corresponds to the external pressure p₂. The following then applies:

p₂*(V₁−V_{b})=n₁*R*T

Equating the two preceding equations gives the drawn liquid volume

V_{b}=V₁*(1−p₁/p₂).

However, this only applies if no air enters the blood culture bottles before or during filling. If all the air from a volume V_{S}, for example, from the tube connecting the inflow opening or inflow line to the cannula through which the liquid is drawn into the blood culture bottle, is drawn into the bottles before the liquid, less liquid enters the blood culture bottle as a result. The actual filling quantity V_{b}′ of the bottles is then obtained by the following considerations. The pressure in the bottle is increased by the air from the tube to

p₁′=(p₁*V₁+p_{S}*V_{S})/V₁

Therefore, the following applies to the actual filling quantity

V_{b}′=V₁*(1−p₁/p₂)−V_{S}*(pₛ/p₂)

The difference is

ΔV_{b}=V_{S}*(pₛ/p₂)

This is the difference between the amount of filling and the amount of fluid removed from the blood vessel.

As discussed previously, the internal pressure in the blood culture bottle must be increased in order to be able to reduce the volume of liquid to be drawn or filled. This is done by first equalizing the pressure between the blood culture bottle and the reservoir. Only then is drawing performed and the preceding equations apply, but with altered internal pressure in the blood culture bottle. For the calculation of the volume of the reservoir Vₕ the following applies to begin with:

pₕ*Vₕ=nₕ*R*T

After pressure equalization, the same pressure pₓ prevails in both the reservoir and the blood culture bottle, and the following applies:

pₓ=(p₁*V₁+pₕ*Vₕ)/(V₁+Vₕ)

Substituting this preceding equation into the equation for the drawn fluid volume the result is

V_{b}=V₁*(1−p₁/p₂)

the result is

V_{b}=V₁*(1−(p1*V1+ph*Vh)/(p₂*(V1+Vh)).

Transforming to V_{b} with p₂=pₕ, which means that the pressure in the reservoir corresponds to the ambient (air) pressure, results in

Vₕ=(V₁²/V_{b})*(1−p₁/p₂)−V₁.

In this way, a suitable volume for the reservoir can be calculated for the drawing of a given volume of liquid V_{b}. Again, note that V_{b} is not the actual amount of liquid that will end up in the bottle if some amount of gas is drawn into the bottle from a volume V_{S} ahead of the liquid (see above). In this case, V_{b} is the volume of liquid taken from the patient in the hospital or from a beaker in the laboratory, and V_{b}′ corresponds to the volume of liquid drawn into the blood culture bottle. If pₛ is equal to p₂, the difference of V_{b} and V_{b}′ is equal to the sum of the internal volume of the connecting tube between the patient or the beaker and the blood culture bottle and the lines for the liquid in the reservoir. Under these circumstances, Vₕ then results in

Vₕ=V₁²/(V_{b}′+V_{S})*(1−p₁/p₂)−V₁

Based on the above calculations and taking into account any deviations of the idealized calculations from the actual conditions as well as manufacturing tolerances of mass-produced blood culture bottles, the volume of the reservoir is selected in the context of the present invention, for example, as follows.

| V_{b}′ (ml) | 5 | 7.5 | 10 |
|---|---|---|---|
| Vₕ orange (cm³) | 122.80 ± 8.90 | 70.60 ± 5.27 | 44.50 ± 3.48 |
| Vₕ green (cm³) | 163.17 ± 10.96 | 94.35 ± 7.75 | 59.93 ± 4.68 |

Legend:
V_{b}′: Target volume to be drawn
Vₕ orange: Volume of the reservoir for filling blood culture bottles “orange”
Vₕ green: Volume of the reservoir for filling blood culture bottles “green”

### BRIEF DESCRIPTION OF THE DRAWINGS

The invention is explained in more detail below with reference to an example of an embodiment shown in the drawing. Therein,

FIG. 1 shows a longitudinal section through a device according to the invention,

FIG. 2 shows a perspective view of the longitudinal section according to FIG. 1,

FIG. 3 shows a schematic representation of the device according to the invention during pressure equalization and

FIG. 4 shows a schematic representation of the device according to the invention after pressure equalization.

### DETAILED DESCRIPTION

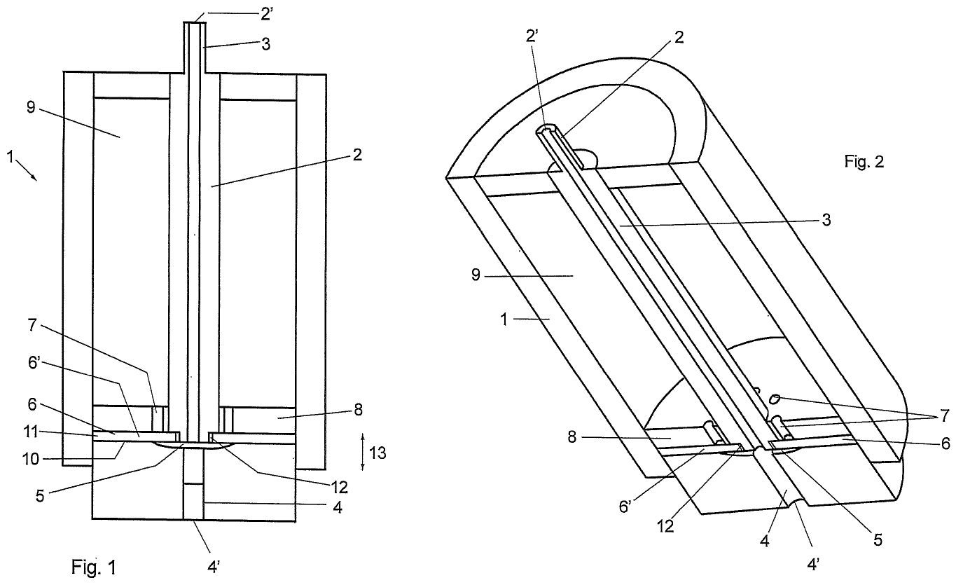

In FIG. 1, a device according to the invention for connecting a cannula to a container being under reduced pressure is generally designated by the reference sign number 1. The device 1, also referred to as adapter has an inflow line 2 with an inflow opening 2′ and a connection 3 for a cannula not shown in FIGS. 1 and 2 or a connecting tube for a cannula. Furthermore, the device 1 has an outflow line 4 with an outflow opening 4′, to which a hollow mandrel not shown in FIGS. 1 and 2 can be connected for piercing a blood culture bottle. A chamber 5 is arranged between the inflow line 2 and the outflow opening 4′, which has an exceedingly small volume and connects the inflow line 2 with the outflow line 4 in a sealing manner. The inflow line 2, the chamber 5 and the outflow line 4 form a fluidic connection between the inflow opening 2′ and the outflow opening 4′.

With the interposition of a valve 6, which in this example is essentially formed by a valve closure member 6′ covering a plurality of holes 7 in a perforated plate 8, a reservoir 9 is connected to the chamber 5, in which a certain amount of gas is preferably present at atmospheric pressure. The volume of the reservoir 9 is, for example, 70 cm³. The perforated plate forms both a wall of the chamber 5 and a wall of the reservoir 9.

Furthermore, it can be clearly seen in FIG. 1 that the perforated plate 8 and a wall 10 of the chamber 5 opposite the perforated plate 8 bear against the valve closure member 6′ at its edge 11 in a clamping manner and thus support the valve closure member 6′. In the area of the valve hole 12, the wall 10 of the chamber 5 opposite the perforated plate 8 is spaced from the valve closure member 6′ to allow movement of the valve closure member in the sense of the double arrow 13.

In FIG. 2, in which the same parts are provided with the same reference numerals, which incidentally also applies to all other figures, it can be seen that the holes 7 in the perforated plate 8 are covered by the valve closure member 6′ on the chamber 5 side in the closed position. Further, it can be seen that the perforated plate 8 centrally supports the inflow line 2 and the reservoir 9 surrounds the inflow line 2.

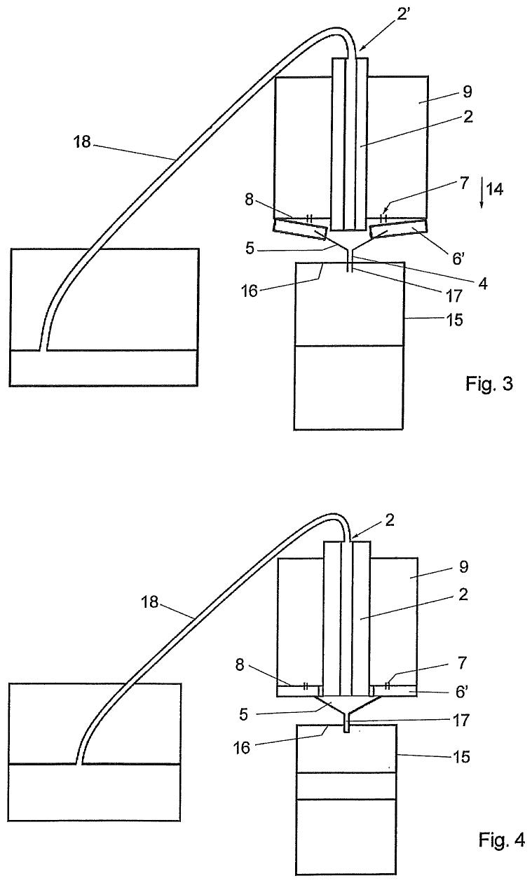

In the schematic representation according to FIG. 3, it can be seen that the valve closure member 6′ is deflected in the direction of arrow 14 by the effect of negative pressure or pressure reduced relative to atmospheric pressure in the container 15, for example a blood culture bottle 15, the closure 16 of which is pierced by a hollow mandrel 17, so that a certain amount of gas is drawn through the holes 7 from the reservoir 9 via the chamber 5 and the outflow line 4 into the container 15. Pressure equalization between chamber 5 and container 15 is done more or less complete, although the resistance or restoring tendency of the valve closure member 6′ will not permit perfect pressure equalization due to its material properties. However, the volumes are selected so that the negative pressure in the container 15 is matched to the level of the atmosphere or to the pressure in a blood vessel of a patient to such an extent that only a limited volume of blood is drawn into the blood culture bottle 15. A cannula or a connecting tube to the cannula is designated by the reference numeral 18. A patient's blood vessel is not shown and is only illustrated by a vessel containing a fluid.

In FIG. 4, the valve closure member 6′ is again shown in the undeflected closed position abutting the holes 7 in the perforated plate 8, and the chamber 5 is filled with liquid. The amount of liquid, in the case of use of the device according to the invention blood, is a dead volume, so that the chamber 5 is designed with the smallest possible volume.

1-21. (canceled)

22. A device for connecting a cannula to a container under reduced pressure, comprising:
an inflow opening configured to connect to one of the cannula and a connecting tube leading to the cannula;
an outflow opening configured to connect with a hollow mandrel configured to pierce a closure of the container; and
a tight fluidic connection of the inflow opening with the outflow opening;
wherein a reservoir that contains a predetermined quantity of gas and that is separate from the fluidic connection is connected to the fluidic connection with the interposition of a valve.

23. The device according to claim 22, wherein:
the container is a blood culture bottle; and
the valve opens towards the fluidic connection.

24. The device according to claim 22, wherein the outflow opening is integrally connected to the hollow mandrel.

25. The device according to claim 22, wherein the quantity of gas in the reservoir is at atmospheric pressure and occupies a volume of 40 cm³ to 100 cm³.

26. The device according to claim 22, wherein the valve is switchable by a differential pressure prevailing between the reservoir and the fluidic connection.

27. The device according to claim 22, wherein the valve is openable by a negative pressure prevailing in the fluidic connection.

28. The device according to claim 22, wherein the valve is closable by an overpressure prevailing in the fluidic connection.

29. The device according to claim 22, wherein a chamber is arranged on a side of the valve that is connected to the fluidic connection, so that an overpressure prevailing in the chamber acts on a valve closure member of the valve in a closing direction.

30. The device according to claim 29, wherein the reservoir has a perforated plate with at least one hole as a reservoir wall and the at least one hole is covered by the valve closure member of the valve in a closed position of the valve on a side facing to the chamber.

31. The device according to claim 30, wherein the perforated plate is circular.

32. The device according to claim 22, wherein the reservoir is formed as an annular chamber surrounding the fluidic connection.

33. The device according to claim 32, wherein the perforated plate supports the fluidic connection.

34. The device according to claim 29, wherein the valve closure member is designed as a circular, deformable disc with a valve hole.

35. The device according to claim 34, wherein the valve hole is centrally formed.

36. The device according to claim 30, wherein:
the perforated plate and a wall of the chamber opposite the perforated plate bear against the valve closure member at an edge thereof; and
in a region of the valve hole, a wall of the chamber opposite the perforated plate is spaced apart from the valve closure member.

37. The device according to claim 30, wherein the perforated plate and the wall of the chamber opposite the perforated plate bear against the valve closure member at an edge thereof in a clamping manner.

38. The device according to claim 29, wherein the valve closure member is made of a material selected from the group consisting of rubber, silicone and polyolefins.

39. The device according to claim 22, wherein the valve is configured to open at a negative pressure of the fluidic connection with respect to the reservoir of 0.3 bar to 0.8 bar.

40. The device according to claim 22, wherein the valve is designed to close at an overpressure of the fluidic connection with respect to the reservoir of 0.1 bar to 0.4 bar.

41. A method of connecting a cannula to a container under reduced pressure, comprising:
providing a connecting device for connecting the cannula to the container being under reduced pressure, in particular a connecting device according to claim 22;
connecting the connecting device to the cannula and to the container that is under reduced pressure, thereby establishing a fluidic connection from the cannula to the container; and
drawing a quantity of gas from a reservoir of the connection device, which is separate from the fluidic connection, into the container, said drawing being effected by a negative pressure prevailing in the container relative to the reservoir.

42. The method according to claim 41, wherein the container is a blood culture bottle.

43. The method according to claim 41, wherein said drawing of the quantity of gas from the reservoir into the container is carried out until one of a complete and a partial pressure equalization between the reservoir and the container.

44. The method according to claim 41, wherein said drawing of the gas quantity from the reservoir into the container takes place via a valve arranged between the reservoir and the fluidic connection.

45. The method according to claim 41, wherein the reservoir is filled with a predetermined quantity of gas, which is dimensioned in such a way that the pressure prevailing in the container is increased by 30%-100%.

46. The method according to claim 41, wherein a predetermined amount of gas is provided in the reservoir at atmospheric pressure and having a volume of 40 cm³ to 100 cm³.

47. The method according to claim 41, wherein, after the amount of gas is drawn from the reservoir into the container, a liquid is drawn from the cannula into the container via the fluidic connection.

48. The method according to claim 47, wherein said drawing of the liquid from the cannula is performed by a negative pressure prevailing in the container with respect to the liquid pressure in the cannula.
